(19) United States
(12) Patent Application Publication
(10) Pub. No.: US 2003/0100605 A1
(43) Pub. Date: May 29, 2003

(54) Methods of treating cancer with angiogenesis inhibitors

(76) Inventor: Stephan A. Grupp, Hovertown, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/146,612

(22) Filed: May 15, 2002

Related U.S. Application Data
(60) Provisional application No. 60/290,921, filed on May 15, 2001.

(51) Int. Cl. A61K 31/336

(52) U.S. Cl. 514/475

(57) ABSTRACT

Disclosed are methods of treating cancer utilizing angiogenesis inhibitors as an adjunct to high-dose therapy and stem cell rescue.

### CROSS REFERENCE TO RELATED PATENT APPLICATION

This application claims the benefit of U.S. Provisional Application No. 60/290,921 filed on May 15, 2001, the entire disclosure of which is incorporated by reference herein.

### FIELD OF THE INVENTION

The present invention relates to administration of angiogenesis inhibitors (AIs) in conjunction with transplantation of stem cells as a means of treating cancer. More specifically, this invention provides methods to treat cancer patients with therapeutically effective amounts of AIs as an adjunct to high dose therapy and stem cell rescue, at a point in the patient's therapy when disease has been reduced as much as chemotherapy allows (referred to herein as minimal residual disease).

### BACKGROUND OF THE INVENTION

A number of background publications are referenced in this application in order to more fully describe the state of the art to which this invention pertains. Full citations for these references are provided at the end of the specification. The entire disclosure of each of these publications is incorporated by reference herein.

Chemotherapy is a well-established and generally appropriate method of treating cancer. Intensified chemotherapy has improved survival rates for some patients with high-risk solid tumors. This has included patients with relapsed Hodgkin's disease as well as pediatric patients with chemotherapy-responsive malignancies such as Ewing's sarcoma and neuroblastoma (1, 2). Increases in dose-intensification have been facilitated by improved supportive care, use of hematopoietic growth factors, use of bone marrow (BM) as stem cell support and, more recently, use of peripheral blood progenitor cells to allow rapid return of marrow function after myeloablative chemotherapy, an approach that has been termed “megatherapy” or high-dose chemotherapy with stem cell rescue. However, even the most dose-intensified approaches are still limited by the risk of relapse after the procedure.

One major risk factor for relapse after high-dose chemotherapy with stem cell rescue is the presence of bulk disease prior to the stem cell procedure. Even for patients who are clinically determined to be in complete remission, however, relapse is still a concern. For these patients, relapse may arise from minimal residual disease within the patient or tumor inadvertently reinfused with the stem cell product. There is indirect evidence suggesting that reinfused tumor may sow the seeds for later relapse. In neuroblastoma, gene-marked tumor cells infused with BM used to support high-dose chemotherapy can be detected at sites of subsequent relapse (3, 4). In patients with lymphoma who undergo stem cell transplantation, molecular detection of tumor in the stem cell product is a predictor for relapse (5). However, no trial reported to date has shown an advantage for patients who receive stem cell products processed in an attempt to remove or decrease infused tumor.

Anti-angiogenic therapy has shown promising results in animal studies (6-10) and has been relatively nontoxic in early human clinical trials. Phase I development of AIs has focused on treatment of relapsed patients with bulk disease. Because these drugs have their effect at the level of normal (nontransformed) endothelium (11), clonal evolution or induced chemotherapy resistance within the tumor should not affect response to AIs (10).

Folkman first reported that analogues of fumagillin are potent inhibitors of endothelial cell proliferation, leading to the discovery of TNP-470 (20). TNP-470, which is currently in clinical trials, is active in mouse xenograft models in bulk disease, with even greater efficacy apparent in the setting of minimal residual disease (12-14).

Cohn and associates reported that increased vascularity in neuroblastoma is associated with aggressive disease and poor outcome (21), suggesting that there may be a role for AIs in the treatment of advanced disease. Since that time, several studies have explored the use of TNP-470 in animal models of malignant tumors. TNP-470 seems to be most effective when used in the setting of minimal disease burden (12), especially when used prior to objective evidence of disease establishment (14). Studies have also found that TNP-470 first administered ten days after inoculation of mice with two different neuroblastoma cell lines decreased the primary tumor volume and the size and number of lymph node and liver metastases (22). Similar results have been seen with other xenograft models using malignant human cell lines such as choriocarcinoma, ovarian cancer and endometrial cancer (23).

Metastatic solid tumors of childhood have been historically difficult to treat, especially high-risk neuroblastoma. Surgery plus conventional chemoradiotherapy has provided only 20% survival at best (17). Addition of autologous BM transplantation and biotherapy with 13-cis-retinoic acid improved 3-year event-free survival to approximately 40% in a Children's Cancer Group Phase III randomized trials (2). Further dose-intensification with tandem transplantation and use of peripheral blood progenitor cells as stem cell support has provided evidence of further improvement in event-free survival (18, 19), but this approach has not yet been validated in a Phase III study. Despite these relative improvements in outcome, the majority of children with high-risk neuroblastoma still experience relapse. Chemotherapy dose-intensification has reached its limit.

Thus, there is a need to provide improved therapies for treating cancer patients and a particular need for optimized methods designed to treat patients having severe types of cancer associated with poor response to currently utilized therapeutic intervention and poor prognosis.

### SUMMARY OF THE INVENTION

The present invention addresses the need for improved therapeutic approaches for the treatment of patients having severe types of cancer. In accordance with one aspect of the present invention, a cancer treatment method is provided in which anti-angiogenic agents are administered during and after recovery from high-dose therapy with stem cell transplant, thereby decreasing the risk of relapse.

According to another aspect of the present invention, methods are provided for the treatment of cancer patients with AIs in conjunction with high-dose therapy, which therapy involves the administration of at least one chemotherapeutic agent to a cancer patient at levels sufficient to effect total ablation of bone marrow-derived cells, together with stem cell transplantation. According to a further aspect of the present invention, methods are provided for the treatment of cancer patients with AIs in conjunction with high-dose therapy, which therapy involves the administration of radiation treatment to a cancer patient at levels sufficient to effect total ablation of bone marrow-derived cells, together with stem cell transplantation.

According to still another aspect of the present invention, methods are provided for the treatment of cancer patients with AIs in conjunction with high-dose therapy, which therapy involves the combined administration of radiation treatment and at least one chemotherapeutic agent to a cancer patient at levels sufficient to effect total ablation of bone marrow-derived cells, together with stem cell transplantation.

According to yet another aspect of the present invention, methods are provided for the treatment of cancer patients with AIs in conjunction with high-dose therapy, which therapy involves the combined administration of localized radiation treatment and at least one chemotherapeutic agent to a cancer patient at levels sufficient to effect total ablation of bone marrow-derived cells, together with stem cell transplantation.

According to a further aspect of the present invention, methods are provided for the treatment of pediatric cancer patients with AIs as an adjunct to high-dose therapy with stem cell transplantation, wherein the therapy has been optimized for pediatric cancer patients.

This combined therapeutic approach may extend the duration of remission from disease and may potentially facilitate long-term complete remission. More specifically, the method of the present invention is expected to improve the prognosis of patients afflicted with the more severe types of cancer, including: relapses, advanced solid tumors, other high-risk solid tumors, and pediatric malignancies such as Ewing's sarcoma and neuroblastoma.

### BRIEF DESCRIPTION OF THE DRAWINGS

FIG. 1 is a bar graph of colony growth vs. TNP-470 dose. The graph shows that TNP-470 at high concentrations is inhibitory for in-vitro colony formation from BM hematopoietic progenitors. BM cells were cultured in duplicate in standard methylcellulose medium supplemented with growth factors at the indicated concentrations of TNP-470. These results are representative of three experiments.

FIG. 2 is a set of two bar graphs showing the percentage of mice surviving treatment vs. the treatment administered, i.e., stem cell transplant alone, or stem cell transplant in conjunction with TNP-470. Overall survival after stem cell rescue at all dose levels (see Table 3 for details of dosing) is indicated on the right. Survival after stem cell rescue at dose level 2 (a single dose of TNP-470 on the day of stem cell rescue) is also separately indicated. These data represent the average lymphoid engraftment of at least 3 experiments per dose level, with 5-8 mice per group. The differences between treatment and control are not statistically significant.

FIG. 3 is a photograph of an electrophoresis gel showing the PCR analysis of transgene (Tg) expression in BM, spleen and peripheral blood. A) Genomic DNA was isolated from BM cells and splenocytes depleted of red blood cells. The transgene was then detected by PCR amplification of the Vκ gene. B) T cells were flow-sorted from peripheral blood or column-purified from spleen and then subjected to PCR to detect the transgene. Tail DNA from the Tg− recipient provided the negative control, while BM, spleen and splenic T cells from a Tg+ donor provided positive controls for expression of the transgene.

### DETAILED DESCRIPTION OF THE INVENTION

In accordance with the present invention, improved therapeutic regimens are provided for the treatment of patients with cancer which involve administration of angiogenesis inhibitors as an adjunct to certain known anticancer agents.

As used herein, the term “cancer” refers to an abnormal growth of tissue resulting from uncontrolled progressive multiplication of cells and serving no beneficial physiological function. Examples of cancers that can be treated according to a method of the present invention include, without limitation, sarcomas, blastomas, and carcinomas such as: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, meningeal carcinomatosis (which is most commonly associated with disseminated breast or lung cancer), ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular cancer, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Examples of hematologic malignancies that can be treated according to a method of the present invention include: acute myeloid leukemia (AML), chronic myeloid leukemia (CML), acute lymphocytic leukemia (ALL), chronic lymphocytic leukemia (CLL), multiple myeloma, non-Hodgkin's lymphoma (NHL), Hodgkin's disease and lymphoma (HD), prolymphocytic leukemia (PLL), and myelodysplastic syndrome (MDS).

The term “anticancer agent” as used herein denotes a chemical compound or electromagnetic radiation (especially, X-rays) that is capable of modulating tumor growth or metastasis. When referring to use of such an agent with an angiogenesis inhibitor, such as, for example, TNP-470, the term refers to an agent other than the angiogenesis inhibitor. Unless otherwise indicated, this term can include one, or more than one, such agents. Thus, the term “anticancer agent” encompasses the use of one or more chemotherapeutic substance and/or electromagnetic radiation in practicing the methods of the invention.

Suitable classes of anticancer agents, and their proposed mechanisms of action, are described below:

1. Alkylating agent: a compound that donates an alkyl group to nucleotides. Alkylated DNA is unable to replicate itself and cell proliferation is inhibited. Examples of such compounds include, but are not limited to, busulfan, coordination metal complexes (such as carboplatin, oxaliplatin, and cisplatin), cyclophosphamide (cytoxan), dacarbazine, ifosfamide, mechlorethamine (mustargen), and melphalan;

2. Bifunctional alkylating agent: a compound having two labile methanesulfonate groups that are attached to opposite ends of a four carbon alkyl chain. The methanesulfonate groups interact with, and cause damage to DNA in cancer cells, preventing their replication. Examples of such compounds include, without limitation, chlorambucil and melphalan;

3. Non-steroidal aromatase inhibitor: a compound that inhibits the enzyme aromatase, which is involved in estrogen production. Thus, blockage of aromatase results in the prevention of the production of estrogen. Examples of such compounds include anastrozole and exemstane;

4. Immunotherapeutic agent: an antibody or antibody fragment that targets cancer cells that produce proteins associated with malignancy. Exemplary immunotherapeutic agents include Herceptin which targets HER2 or HER2/neu cell surface receptors expressed at high levels in about 25 to 30 percent of breast cancers; monoclonal antibodies such as C225, and anti-CD20 which triggers apoptosis in B cell lymphomas;

5. Nitrosourea compound: inhibits enzymes that are needed for DNA repair. These agents are able to travel to cross the blood-brain barrier and, therefore, may be used to treat brain tumors, as well as non-Hodgkin's lymphomas, multiple myeloma, and malignant melanoma. Examples of nitrosureas include carmustine and lomustine;

6. Antimetabolite: a class of drugs that interfere with DNA and ribonucleic acid (RNA) elongation. These agents are cell cycle phase specific (S phase) and are used to treat chronic leukemias as well as tumors of breast, ovary and the gastrointestinal tract. Examples of antimetabolites include 5-fluorouracil, methotrexate, gemcitabine (GEMZAR), cytarabine (Ara-C), and fludarabine.

7. Antitumor antibiotic: a compound having antimicrobial and cytotoxic activity. Such compounds also may interfere with DNA by chemically inhibiting enzymes and mitosis or altering cellular membranes. Examples include, but certainly are not limited to bleomycin, dactinomycin, daunorubicin, doxorubicin (Adriamycin), and idarubicin;

8. Mitotic inhibitor: a compound that can inhibit mitosis (e.g., tubulin binding compounds) or inhibit enzymes that prevent protein synthesis needed for reproduction of the cell. Examples of mitotic inhibitors include taxanes such as paclitaxel and docetaxel, epothilones, etoposide, vinblastine, vincristine, and vinorelbine;

9. Radiation therapy: includes but is not limited to X-rays or gamma rays which are delivered from either an externally supplied source such as a beam or by implantation of small radioactive sources.

10. Topoisomerase I inhibitors: agents that interfere with topoisomerase activity thereby inhibiting DNA replication. Such agents include, without limitation, CPT-11 and topotecan.

11. Hormonal therapy: includes, but is not limited to anti-estrogens, such as Tamoxifen, GnRH agonists, such as Lupron, and Progestin agents, such as Megace.

Other types of anticancer agents, for example, leuocovorin. kinase inhibitors, such as Iressa and Flavopiridol, and analogues of conventional chemotherapeutic agents, such as taxane analogues and epothilone analogues can be utilized in carrying out the method of the present invention. Retinoids, such as Targretin, can also be utilized in the method of the invention. Signal transduction inhibitors that interfere with farnesyl transferase activity and chemotherapy resistance modulators, e.g., Valspodar can also be employed.

High-dose therapy (also known as high-dose chemotherapy, megatherapy or maximal dose-intensity therapy) is a recently developed therapeutic approach implemented to provide more efficacious treatment for patients having severe cancers. In brief, high-dose therapy involves the treatment of a patient with one of the following: high-dose radiation, high-dose chemotherapy, or a combination of high-dose radiation and chemotherapy to effect total ablation of bone marrow cells. While this treatment improves the likelihood of the elimination of circulating cancer cells, it necessitates that patients receive hematopoietic stem cell transplants (HSCT) to reconstitute their population of bone marrow derived circulating cells, such as, for example, B and T lymphocytes and granulocytes. Although the implementation of high-dose therapy has improved the prognosis for some patients with severe types of cancer, the long-term survival rate for many patients is still low. For example, approximately 30% of leukemia patients relapse after allogeneic bone marrow transplantation using total body irradiation-based preparative regimens, indicating that the treatment may be suboptimal (26).

High-dose radiation, or total body irradiation, involves the administration of appropriate levels of irradiation from a high-energy source in a clinical setting. Doses of fractionated total body irradiation generally range from a prescription of approximately 10-14 Gy (Gray) delivered using a high-energy source (27). The appropriate dose for total body irradiation is determined based on a number of factors, including, for example, the type of cancer manifested, the patient's condition, and history of previous treatments and the judgment of the attending physician. This treatment is usually given over 3-4 days in 6-8 fractions, but may be given in a single dose (fraction).

High-dose therapy can also involve the administration of chemotherapy agents to effect total ablation of bone marrow cells. A variety of different chemotherapeutic agent cocktails have been described for use in high-dose chemotherapy. Such cocktails can include, by way of example, high dose combinations of ICE (ifosfamide, carboplatin, and etoposide), CEC (carboplatin, etoposide, cyclophosphamide), CEM (carboplatin, etoposide, melphalan), and TC (thioTEPA and cyclophosphamide) as previously described (2, 28-33, 38, 40-42). High-dose chemotherapy can be administered in a single bolus or over the course of multiple cycles. The choice of chemotherapeutic agents and the mode of administration are likewise determined on the basis of the factors noted above for high-dose radiation, according to established practice in the field of oncology.

High-dose chemotherapy regimens have also been developed for the pediatric population that have been adjusted to optimize survival rate of children having a variety of different cancers, while minimizing transplant-related complications. Such regimens include the administration of cocktails containing adjusted doses of oral busulfan, thiotepa, and cyclophosphamide for the treatment of children with advanced hematologic malignancies (34) or multiple courses of high-dose chemotherapy and/or myeloablative therapy for the treatment of children with AML (reviewed in 35). Since toxicity associated with therapeutic intervention is particularly pronounced in pediatric patients, the availability of risk-tailored therapy for such patients provides a clinician with useful guidelines for therapeutic intervention. Such risk-tailored therapy is based on cytogenetic risk stratification, promptness of remission induction, and identification of distinct clinical subgroups such as children with Downs' Syndrome (reviewed in 35).

High-dose therapy can also involve the initial treatment of a patient with a combination of high-dose radiation and chemotherapy to effect total ablation of bone marrow cells. Doses of fractionated total body irradiation generally range from a prescription of approximately 10-14.4 Gy delivered using a high-energy source (26). Examples of chemotherapeutic agents that can be used in conjunction with high dose radiation include, but are not limited to, cyclophosphamide with or without etoposide, cytosine arabinoside, busulfan, melphalan or a combination cocktail of cisplatin, etoposide, and ifosfamide.

Treatment utilizing high-dose therapy comprised of radiation and chemotherapy can involve localized irradiation of specific regions of the body in which a tumor, for example, resides. Higher intensity doses of irradiation can be used to target specific regions of the body, due to the localized nature of cellular toxicity, and generally range from a prescription of approximately 18-54 Gy delivered using a high-energy source. Brain irradiation, for example, can be achieved by the application of a 30-50 Gy+/−10 Gy boost in patients with brain metastases (36).

As indicated above, since all forms of high-dose therapy effect total ablation of bone marrow cells, each necessitates that a patient receive an HSCT in order to survive.

The terms “hematopoietic stem cell transplant”, “stem cell transplant”, and “bone marrow transplant” as used herein denote the transfer of a population of bone marrow- or peripheral blood-derived pluripotent cells from a patient to himself or from a donor to a recipient for the purposes of repopulating multi-lineage hematopoietic cells in the patient (39, 52-55).

The terms “stem cell rescue”, “hematopoietic stem cell rescue”, “stem cell reconstitution”, “hematopoietic stem cell reconstitution” and “engraftment” as used herein denote successful repopulation of multi-lineage hematopoietic cells in a recipient following stem cell transplant.

Such stem cell, or bone marrow cell, transplants reconstitute the patient's population of bone marrow derived circulating cells, including hematopoietic and immune cells. Both allogeneic and autologous bone marrow/peripheral blood progenitor cell transplantations have been performed successfully, with similar reconstitution of lymphocyte subsets achieved. Where the goal is solely the support of the patient through the ablation of the marrow, stem cells from the patient himself, or autologous transplantation, may be employed. For these patients, peripheral blood stem cell transplantation may decrease the time required for full reconstitution, recovery and acquisition of lymphocyte function as compared to recovery following bone marrow transplantation (reviewed in 37). Hospital stays are shorter, the infection rate is lower, transfusion needs decrease, and mortality is improved with use of stem cells over marrow. Rescue with 2-2.5×10⁶ CD34+ cells/kg has been shown to reconstitute the total number of B cells and T cells to normal levels within 2-4 months post-autologous peripheral stem cell transplantation (APSCT). It is noteworthy that the normal ratio of T cell subsets remains imbalanced even at a year post-APSCT. This imbalance renders patients more susceptible to infections for a prolonged period of time post-transplant (37).

Stem cell grafts, mobilized by treatment with granulocyte colony stimulating factor (G-CSF) or granulocyte-monocyte colony stimulating factor (GM-CSF) have also been used successfully to demonstrate that reconstitution in the presence of these cytokines can enhance the number and function of anti-tumor effector cells in a stem cell transplant, without impairing hematologic reconstitution (38). Treatment with other hematopoietic growth factors and cytokines, including, but not limited to G-CSF and GM-CSF, has also been shown to enhance the activity of anti-tumor effector cells (reviewed 39).

Treatment of cancer patients with angiogenesis inhibitors in conjunction with high-dose therapy, wherein the therapy regimen includes utilization of autologous stem cell transplantation, is within the scope of this invention, as is treatment with angiogenesis inhibitors in conjunction with high-dose therapy, wherein the therapy regimen includes utilization of allogeneic stem cell transplantation.

The choice of a therapy regimen that includes either an autologous or allogeneic stem cell transplant is determined on the basis of a number of factors including, but not limited to the type of cancer manifested, the condition of the patient, and established practice in the field of oncology (reviewed 39).

In carrying out stem cell transplantation in accordance with this invention, it is advantageous for the stem cell graft to be mobilized by treatment with cytokines to enhance the number of stem and progenitor cells, as well as the activity of anti-tumor effector cells. These stem cells may also be purged of contaminating tumor cells or other cell populations by the process of CD34 selection (43-45).

Given post-stem cell infusion in the setting of high-dose therapy with stem cell rescue, AIs have the potential to lessen the risk of relapse from minimal residual disease, whether within the patient or infused with the stem cell support.

Relapse after maximal dose-intensity therapy may, in part, result from contamination of the stem cell product with tumor cells (3, 4). Whether relapse results from reinfused tumor cells or cells remaining in the patient, most patients are in a state of minimal residual disease after transplant. This provides a clinical situation in which the use of AIs may be most effective.

Moreover, since AIs are relatively nontoxic (46, 47) and cancer cells are not known to develop resistance to AIs (reviewed in 48), the administration of AIs to patients for prolonged duration following high-dose therapy together with stem cell rescue is feasible.

AIs suitable for use in the present invention include natural and synthetic inhibitors of the proteins known to activate endothelial cell growth and movement (reviewed in 49-51). Those proteins include, for example, vascular endothelial growth factor (VEGF), acidic and basic fibroblast growth factors (aFGF and bFGF), angiogenin, epidermal growth factor (EGF), scatter factor (SF), placental growth factor (P1GF), interleukin-8, transforming growth factor-alpha (TGF-α) and TGF-β, angiopoietin-1, and tumor necrosis factor alpha (TNF-α). Some of the known, naturally occurring inhibitors of angiogenesis are angiostatin, endostatin, interferons, platelet factor 4 (PF4), thrombospondin, fumagillin, transforming growth factor beta, 16 Kd fragment of prolactin, and tissue inhibitor of metalloproteinase-1, -2, and -3 (TIMP-1, TIMP-2 and TIMP-3).

A preferred class of AIs for use in the present invention includes fumagillin derivatives, including pharmaceutically acceptable salts, having the formula (I):

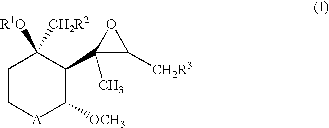

wherein:

R¹ represents hydrogen;

R² represents halogen, N(O)ₘR^{a}R^{b}, S(═O)ₙR^{a}, N⁺R^{a}R^{b}R^{c}X⁻, or S⁺R^{a}R^{b}X⁻;

R^{a}, R^{b}, and R^{c} each being independently selected from the group consisting of hydrogen, an unsubstituted or substituted hydrocarbon, and an unsubstituted or substituted heterocyclic group;

alternatively, R¹ and R² together represent a chemical bond;

R³ represents an unsubstituted or substituted 2-methyl-1-propenyl or isobutyl group;

A represents >CH—OR⁴, >CH—NR⁵R⁶, or >C═O

R⁴ representing hydrogen; unsubstituted or substituted acyl; unsubstituted or substituted alkyl; unsubstituted or substituted aryl; unsubstituted or substituted carbamoyl; unsubstituted or substituted benzenesulfonyl; unsubstituted or substituted alkylsulfonyl; unsubstituted or substituted sulfamoyl; unsubstituted or substituted alkoxycarbonyl; or unsubstituted or substituted phenoxycarbonyl;

R⁵ and R⁶ each independently representing hydrogen, unsubstituted or substituted acyl; unsubstituted or substituted alkyl; unsubstituted or substituted aryl;

unsubstituted or substituted carbamoyl; unsubstituted or substituted alkoxycarbonyl;

or unsubstituted or substituted phenoxycarbonyl;

X⁻ is a counteranion;

m is an integer 0 or 1; and

n is an integer 0, 1, or 2.

The above compound of formula (I) has asymmetric centers in its molecule and is optically active. Its absolute configuration, however, is based on the starting material, fumagillol, and the absolute configuration is consistent with that of fumagillol unless otherwise specified.

The hydrocarbon group represented by R^{a}, R^{b}, or R^{c} in formula I includes straight-chain or branched C₁₋₆ alkyl groups (e.g. methyl, ethyl, propyl, isopropyl, butyl, isobutyl, sec-butyl, pentyl, isopentyl, hexyl, etc.), C₂₋₆ alkenyl groups (e.g. vinyl, allyl, 2-butenyl, methylallyl, 3-butenyl, 2-pentenyl, 4-pentenyl, 5-hexenyl, etc.), C₂₋₆ alkynyl groups (e.g. ethynyl, propargyl, 2-butyn-1-yl, 3-butyn-2-yl, 1-pentyn-3-yl, 3-pentyn-1-yl, 4-pentyn-2-yl, 3-hexyn-1-yl, etc.), C₃₋₆ cycloalkyl groups (e.g. cyclopropyl, cyclobutyl, cyclopentyl, cyclohexyl, etc.), C₃₋₆ cycloalkenyl groups (e.g. cyclobutenyl, cyclopentenyl, cyclohexenyl, cyclohexadienyl, etc.), C₇₋₁₃ aralkyl groups (e.g. benzyl, 1-phenylethyl, 2-phenylethyl, etc.), and C₆₋₁₀ aryl groups (e.g. phenyl, naphthyl, etc.).

The heterocyclic group represented by R^{a}, R^{b}, or R^{c} includes 5- or 6-membered heterocyclic groups containing 1 to 4 hetero-atoms (e.g. nitrogen, oxygen, sulfur, etc.), such as 2-furyl, 2-thienyl, 4-thiazolyl, 4-imidazolyl, 4-pyridyl, 1,3,4-thiadiazol-2-yl, and tetrazolyl. This heterocyclic group may be condensed with a 5- or 6-membered ring which may contain 1 to 3 hetero-atoms (e.g. nitrogen, oxygen, sulfur) other than carbon (e.g. benzene, pyridine, cyclohexane, etc.) to form a condensed bicyclic group (e.g. 8-quinolyl, 8-purinyl, etc.).

When substituted, the hydrocarbon or heterocyclic group represented by R^{a}, R^{b}, or R^{c} may contain 1 to 3 substituents at the possible positions. Examples of such substituent(s) include C₁₋₆ alkyl groups, C₂₋₆ alkenyl groups, C₂₋₆ alkynyl groups, C₃₋₆ cycloalkyl groups, C₃₋₆ cycloalkenyl groups, C₆₋₁₀ aryl groups, amino groups, mono-C₁₋₆ alkylamino groups, di-C₁₋₆ alkylamino groups, azido groups, nitro groups, halogens, hydroxy groups, C₁₋₄ alkoxy groups, C₆₋₁₀ aryloxy groups, C₁₋₆ alkylthio groups, C₆₋₁₀ arylthio groups, cyano groups, carbamoyl groups, carboxy groups, C₁₋₄ alkoxy-carbonyl groups, C₇₋₁₁ aryloxycarbonyl groups, carboxy-C₁₋₄ alkoxy groups, C₁₋₆ alkanoyl groups, halo-C₁₋₆ alkanoyl groups, C₇₋₁₁ aroyl groups, C₁₋₆ alkylsulfonyl groups, C₆₋₁₀ arylsulfonyl groups, C₁₋₆ alkylsulfinyl groups, C₆₋₁₀ arylsulfinyl groups, 5- or 6-membered heterocyclic groups, 5- or 6-membered heterocyclic carbonyl groups, and 5- or 6-membered heterocyclic thio groups.

Referring to the above formula I, the substituent(s) on the unsubstituted or substituted 2-methyl-1-propenyl or isobutyl group represented by R³ include, without limitation, hydroxyl, amino, lower (C₁₋₃) alkylamino (e.g. methylamino, ethylamino, isopropylamino), di-lower (C₁₋₃) alkylamino (e.g. dimethylamino, diethylamino) and a 5- or 6-membered heterocyclic ring containing nitrogen atom (e.g. pyrroridin-1-yl, piperidino, morpholino, piperazin-1-yl, 4-methylpiperazin-1-yl, 4-ethylpiperazin-1-yl), particularly preferred among them being hydroxyl and dimethylamino.

The unsubstituted or substituted acyl group represented by R⁴ may comprise a straight-chain or branched, saturated or unsaturated hydrocarbon group, preferably containing 1 to 20 carbon atoms in the unsubstituted form (e.g. formyl, acetyl, propionyl, isopropionyl, butyryl, pentanoyl, hexanoyl, heptanoyl, octanoyl, nonanoyl, lauroyl, undecanoyl, myristoyl, palmitoyl, stearoyl, arachinoyl, or the like, and the unsaturated analogs), having at least one, preferably one to three substituents each selected from among amino, lower alkylamino (e.g. methylamino, ethylamino, isopropylamino, etc.), di-(lower alkyl)amino (e.g. dimethylamino, diethylamino, etc.), nitro, halogen (e.g. fluorine, chlorine, bromine, iodine, etc.), hydroxyl, lower alkoxy (e.g. methoxy, ethoxy, etc.), cyano, carbamoyl, carboxyl, lower alkoxycarbonyl (e.g. methoxycarbonyl, ethoxycarbonyl, etc.), carboxy-lower alkoxy (e.g. carboxymethoxy, 2-carboxyethoxy, etc.), phenyl which may be unsubstituted or substituted, aromatic heterocyclic group (preferably 5- or 6-membered aromatic heterocyclic group containing one to four hetero atoms each selected from among nitrogen, oxygen, sulfur and so on; e.g. 2-furyl, 2-thienyl, 4-thiazolyl, 4-imidazolyl, 4-pyridyl, etc.) and other substituents, particularly preferred among them being 3-carboxypropionyl and 4-carboxybutyryl; an aroyl group including, without limitation, benzoyl, 1-naphthoyl and 2-naphthoyl, each having at least one, preferably one to three substituents each selected from among C₂₋₆ lower alkyl, such as ethyl or propyl, amino, halogen (e.g. fluorine, chlorine, bromine, etc.), hydroxyl, lower alkoxy (e.g. methoxy, ethoxy, etc.), cyano, carbamoyl, carboxyl and other substituents, 2-carboxybenzoyl being preferred; and a heterocycle-carbonyl group, e.g., 5- or 6-membered rings containing one to four heteroatoms each selected from among nitrogen, oxygen, sulfur and so on. Preferred among others are 2-furoyl, 2-thenoyl, nicotinoyl, isonicotinoyl and imidazole-1-carbonyl. As the substitutent(s) on the heterocycle-carbonyl group there may be mentioned those identified above referring to the substituted aroyl group.

As the alkyl group represented by R⁴, which may be unsubstituted or substituted, there may be mentioned straight or branched C₁₋₂₀ alkyl groups, preferably C₁₋₆, i.e., lower alkyl groups, which may optionally have one to three substituents each selected from among, for example, those substituents mentioned above for the substituted acyl group. This alkyl group may be epoxidized at any optional position. Methyl, ethyl, benzyl and the like are preferred among others.

The aryl group represented by R⁴ includes C₆₋₁₀ aryl groups such as phenyl, naphthyl, or the like. The possible substitutents and the positions thereof are the same as previously mentioned, referring to the unsubstituted or substituted hydrocarbon or heterocyclic groups represented by R^{a}, R^{b}, or R^{c}.

The carbamoyl group, which may be unsubstituted or substituted, represented by R⁴ includes carbamoyl, monosubstituted carbamoyl, and disubstituted carbamoyl. As the substituents, there may be mentioned, for example, lower alkyl (e.g. methyl, ethyl, propyl, butyl, etc.), lower alkanoyl (preferably containing 1 to 6 carbon atoms; e.g. acetyl, propionyl, acryloyl, methacroyl etc.), chloroacetyl, dichloroacetyl, trichloroacetyl, lower alkoxycarbonylmethyl (e.g. methoxycarbonylmethyl, ethoxycarbonylmethyl, etc.), carboxymethyl, amino, phenyl which may be unsubstituted or substituted, naphthyl, benzoyl, and substituents forming, together with the carbamoyl nitrogen atom, cyclic amino groups (e.g. pyrrolidin-1-yl, piperidino, morpholino, piperazin-1-yl, 4-methylpiperazin-1-yl, 4-ethylpiperazin-1-yl, 4-phenylpiperazin-1-yl, imidazol-1-yl, etc.). Preferred among them are chloroacetyl, phenyl, benzoyl and the like.

The substituent of carbamoyl further includes halogenated lower alkyl (e.g. 2-chloroethyl, 2-bromoethyl, 3-chloropropyl), di-lower alkylamino-lower alkyl (e.g. 2-dimethylaminoethyl, 2-diethylaminoethyl, 3-dimethylaminopropyl), lower alkanoyloxy-lower alkanoyl (e.g. acetoxyacetyl, propionyloxyacetyl), lower alkanoylthio-lower alkanoyl (e.g. acetylthioacetyl, propionylthioacetyl), lower alkylthio-lower alkanoyl (e.g. methylthioacetyl, ethylthiopropionyl), arylthio-lower alkanoyl (e.g. phenylthioacetyl, naphthylthioacetyl), aromatic heterocyclicthio-lower alkanoyl (e.g. 4-pyridylthioacetyl, 2-pyridylthioacetyl, 2-benzothiazolylthioacetyl, 2-benzoxazolylthioacetyl, 2-benzoimidazolylthioacetyl, 8-quinolylthioacetyl, [1-(2-dimethylaminoethyl)tetrazol]-5-ylthioacetyl, 2-methyl-1,3,4-thiadiazol-5-ylthioacetyl, 1-methyl-2-methoxycarbonyl-1,3,4-triazol-5-ylthioacetyl), N-oxy-2-pyridylthio-lower alkanoyl (e.g. N-oxy-2-pyridylthioacetyl), N-lower alkyl-4-pyridiniothio-lower alkanoyl.halide (e.g. N-methyl-4-pyridinoacetyl iodide), dilower alkylamino-lower alkanoyl (e.g. dimethylaminoacetyl, diethylaminoacetyl), ammonio-lower alkanoyl halide (e.g. trimethylammonioacetyl iodide, N-methylpyrrolidinoacetyl chloride), aromatic heterocyclic-carbonyl (e.g. 3-furoyl, nicotinoyl, 2-thenoyl), lower alkoxycarbonyl (e.g. methoxycarbonyl, ethoxycarbonyl), phenoxycarbonyl, chloroacetylcarbamoyl, benzoylcarbamoyl, phenylsulfonyl which may have substituent (e.g. benzensulfonyl, toluensulfonyl) and di(lower alkyl)sulfonio-lower alkanoyl halide (e.g. dimethylsulfonioacetyl iodide).

As the substituent(s) on the unsubstituted or substituted benzenesulfonyl group represented by R⁴, there may be mentioned, for example, lower alkyl (e.g. methyl, ethyl, etc.) and halogen (e.g. fluorine, chlorine, bromine, etc.). One to three such substituents may be present on the benzene ring at any optional position or positions.

As the alkylsulfonyl group represented by R⁴, which may be unsubstituted or substituted, there may be mentioned, among others, C₁₋₆ lower alkylsulfonyl groups, which may have one to three substituents each selected from among, for example, those substituents mentioned above for the substituted alkanoyl group. Preferred among them are methylsulfonyl and ethylsulfonyl.

As the substituent(s) on the unsubstituted or substituted sulfamoyl group represented by R⁴, there may be mentioned, for example, lower alkyl (e.g. methyl, ethyl, propyl, isopropyl, isobutyl, etc.), phenyl and substituted phenyl. The sulfamoyl group may have either one substituent or two substituents which are the same or different.

As the alkoxycarbonyl group represented by R⁴, which may be unsubstituted or substituted, there may be mentioned straight or branched lower alkoxycarbonyl groups, which may optionally have one to three substituents each selected from among those substituents mentioned above, for instance. Preferred among them are methoxycarbonyl, ethoxycarbonyl, propoxycarbonyl, butoxycarbonyl, isobutoxycarbonyl, 1-chloroethoxycarbonyl, and the like.

The substituent(s) on the unsubstituted or substituted phenoxycarbonyl group represented by R⁴ may be the same as those mentioned above for the unsubstituted or substituted benzenesulfonyl group. The phenoxy group may have one to three such substituents at any optional position or positions.

In this specification, the substituent(s) on each “substituted phenyl” group include, among others, lower alkyl (e.g. methyl, ethyl, propyl, butyl, etc.), lower alkoxy (e.g. methoxy, ethoxy, propoxy, etc.), halogen (e.g. fluorine, chlorine, bromine, etc.), haloalkyl (e.g. trifluoromethyl, chloromethyl, bromomethyl, etc.) and nitro. The phenyl ring may have one to five such substituents at any optional position or positions.

Particularly good results in practicing the present invention have been obtained using the compound of formula (II), also known as TNP-470 (TAP Pharmaceutical Products). TNP-470 is an anti-angiogenic agent now in clinical trials. Although it inhibits growth of bone marrow (BM) colony-forming cells in vitro, no significant hematologic toxicity has been seen in Phase I trials.

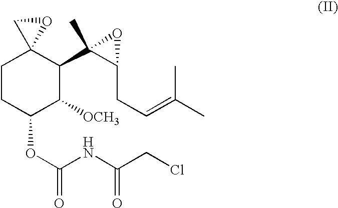

In addition to the above-described compounds and their tautomers, structural isomers, and pharmaceutically acceptable salts, the invention is further directed, where applicable, to solvated as well as unsolvated forms of the compounds (e.g., hydrated forms) having the ability to regulate and/or modulate angiogenesis.

Compounds of formula (I) described herein may be prepared by any process that is known to be applicable to the preparation of chemical compounds. Suitable processes are illustrated, for example, in U.S. Pat. Nos. 5,846,562 and 6,017,954, the entire disclosures of which are incorporated by reference herein. Necessary starting materials may be obtained by standard procedures of organic chemistry. Fumagillin may be purchased from ICN Biomedical Research Products.

The compounds of formula (I) described herein can be administered to a human patient in pure form or in pharmaceutical compositions in combination with at least one suitable carrier or excipient. Techniques for formulation and administration of the compounds of the instant application may be found in “Remington's Pharmaceutical Sciences,” 18^{th} Ed. (1990, Mack Publishing Co., Easton, Pa.) the contents of which are hereby incorporated by reference in their entirety.

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into a solid tumor, often in a depot or sustained release formulation.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Furthermore, one may administer the drug in a targeted drug delivery system, for example, in a liposome coated with tumor-specific antibody. The liposomes will be targeted to and taken up selectively by the tumor.

A preferred route of administration for AIs in the practice of the present invention is intravenous (iv).

Dosage amount and interval may be adjusted individually to provide plasma levels of the AI that are sufficient to maintain the angiogenesis modulating effects, or minimal effective concentration (MEC). The MEC will vary for a given compound but can be estimated from in vitro data; e.g., the concentration necessary to achieve 50-90% inhibition of the angiogenesis using an assay such as the corneal pocket assay (56). Dosages necessary to achieve the MEC will depend on individual characteristics and route of administration. However, HPLC assays or bioassays can be used to determine plasma concentrations.

The expression “therapeutically effective amount”, as used herein, refers to a sufficient amount of a selected AI to provide the desired anticancer effect. The exact amount required will vary from subject to subject, the mode of administration of the compound(s) and the like. A therapeutically effective amount of an AI should not substantially affect engraftment of stem cells in recipients, when administered according to the cancer treatment method described herein, in comparison to those not receiving an AI.

Dosage intervals can also be determined using MEC value.

Compounds should be administered using a regimen that maintains plasma levels above the MEC for 10-90% of the time, preferably between 30-90% and most preferably between 50-90%.

AIs can also be administered at one dose level below the maximum tolerated dose (MTD) as determined in clinical trials.

In cases of local administration or selective uptake, the effective local concentration of the drug may not be related to plasma concentration. The compounds of formula (II) may also be effective biologically far longer than accounted for by serum half-life (57). The compound of formula (II) is generally administered at a dose not to exceed 70 mg/m². The Al can be administered at appropriately spaced intervals, for example, three times per week. Alternatively, the AI can be administered daily or every other day, as a continuous infusion.

In a preferred embodiment of the present invention, the compound of formula I is administered as the AI as a continuous infusion for a duration of two weeks of each month, over a period of 4-6 months, or longer as tolerated by the patient.

Further details regarding suitable routes of administration, dosage, and dosage formulations are provided in the above-mentioned U.S. Pat. Nos. 5,846,562 and 6,017,954.

The following examples are provided to describe the invention in further detail. These examples, which set forth the preferred mode presently contemplated for carrying out the invention, are intended to illustrate and not to limit the invention.

### EXAMPLE I

Abbreviations used herein include: angiogenesis inhibitor, AI; bone marrow, BM; bone marrow transplant, BMT; orally, PO; stem cell transplant SCT; subcutaneously, SQ; total body irradiation, TBI; transgene, Tg; and white blood cell WBC.

Using the methods of the present invention, a reliable system for stem cell transplant has been developed in a mouse model that involves transplanting stem cells from donor mice that express a Tg, which is readily detected by flow cytometry or PCR analysis, into lethally irradiated recipients. The compound of formula (II) (TNP-470) was administered in this setting as the Al, starting on day 0 of transplantation, with minimal toxicity and no excess mortality in the AI-treated group, whether the compound was administered as a bolus dose or as continuous infusion. Both treated and control mice demonstrated reliable multilineage engraftment, as well as normal B cell maturation. Furthermore, engraftment kinetics were not slowed by administration of TNP-470 immediately following infusion of donor stem cells. There was evidence of decreased white blood cell (WBC) in the bolus TNP-470 group compared to controls at day 28 post-treatment. The opposite effect, however, was seen in animals treated continuously with TNP-470, in that the WBC count of TNP-470-treated mice was slightly higher than that of saline-treated mice. Neither of these small effects on WBC was considered clinically significant.

Materials and Methods

Donors, recipients and preparative regimen. Transgenic mice expressing a human IgM Tg in the FVB background were used as the donor source of bone marrow stem cells for transplantation. The FVB strain of inbred mice is frequently utilized in the generation of transgenic animals. Marrow was collected from Tg+ mice from femurs flushed with sterile PBS. Recipients were FVB mice (Jackson Laboratory, Bar Harbor, Me.) or Tg negative littermates of the Tg positive donors, treated in groups of 5-8 animals/intervention. Recipients received total body irradiation (TBI) in an M38-1 Irradiator (Isomedix) at a dose rate of 2.7 Gy/min in a mixed/split fashion with a 3 hour interfraction interval to allow a higher dose of radiation without significant gastrointestinal toxicity. After completion of TBI, mice received stem cells via tail vein injection. The mice were maintained in a temperature controlled humidified area in autoclaved microisolator cages and fed ad libitum and provided acidified water. Mice were assessed three times weekly after stem cell infusion. These studies were approved by the Animal Care and Use Committee of the Children's Hospital of Philadelphia.

Compound (drug). TNP-470 was provided by TAP Pharmaceuticals (Deerfield, Ill.). Prior to use, TNP-470 was reconstituted in sterile saline, aliquoted and stored at −80° C. TNP-470 was used at a dose of 20-100 mg/kg given SQ three times per week beginning at day 0 or at a dose of 10-20 mg/kg/week given by continuous intraperitoneal infusion using an Alzet infusion pump (Alza Co., Palo Alto, Calif.). The continuous TNP-470 infusion began on the day prior to transplant to allow for pump implantation (see below).

Alzet infusion pump placement. Using sterile technique following anesthesia, a 1 cm midline abdominal incision was made and 14 day Alzet micro-osmotic pumps (0.25 μl/hr, model 1002) containing either TNP-470 or saline were placed intraperitoneally. The peritoneum and skin were then secured separately using 4.0 vicryl suture. The animals were allowed to recover overnight and then subjected to TBI and stem cell infusion on the following day.

In vitro bone marrow culture. Light density cells separated by density gradient centrifugation (Lymphocyte Separation Medium, ICN Pharmaceutical, Costa Mesa, Calif.) from normal human BM donors were plated in methylcellulose medium with recombinant cytokines. This medium, MethoCult GF (Stem Cell Technologies, Vancouver, Canada), contains stem cell factor 50 ng/ml, GM-CSF 10 ng/ml, IL-3 10 ng/ml and erythropoietin 3 units/ml. 5×10⁴ BM cells/dish were cultured with TNP-470 at concentrations ranging from 1 mcg/ml to 1 mg/ml, with duplicate cultures at each dose. The plates were scored after 14 days of culture, enumerating colony-forming unit granulocyte/macrophage (CFU-GM), CFU-mix, CFU-erythrocyte and burst-forming unit erythrocyte (CFU-E and BFU-E).

Analysis of engraftment. Engraftment of donor stem cells was demonstrated by both flow cytometry and by polymerase chain reaction (PCR). Following cervical dislocation, BM and splenocytes were collected from recipient mice. Analysis was performed after red cell lysis with NH₄Cl. For flow cytometric analysis of lymphoid engraftment, Tg IgM expressed only in B cells derived from the donor was detected by antibodies recognizing human IgM (RAHM; Jackson ImmunoResearch, West Grove, Pa.) and mouse CD45R (B220, Pharmingen, Torreyana, Calif.) in a two-color protocol on a FACS Caliber cytometer (Becton-Dickinson, Franklin Lakes, N.J.). Splenocytes from untransplanted Tg− and Tg+ mice provided negative and positive controls, respectively. Lymphoid engraftment was defined as the percentage of lymphoid cells in spleen and BM that were B220/RAHM positive. In addition to flow cytometry, genomic DNA from tail snips, blood, BM, and splenocytes was analyzed by PCR for the presence of the transgene, using a procedure and primers previously reported (15). The transgene was detectable by PCR in all donor-derived cells in the recipient, while the tail snips of Tg− mice provided a negative control. Genomic DNA was also isolated from splenic and peripheral blood T cells. Splenic T cells were isolated using the Cellect T isolation column (Biotex, Edmonton, Canada) according to the manufacturer's protocol. Peripheral blood T cells were sorted on the FACS Vantage (Becton-Dickson) after staining with antibodies recognizing mouse CD3 (Pharmingen). Recovery of peripheral blood counts was also analyzed. Blood was collected from cardiac puncture and placed in EDTA tubes (Becton-Dickson). Analysis was then performed with a HemeVet (CDC Technologies) instrument using mouse-specific parameters. Hemoglobin was measured and white blood cells (WBC) and platelets were enumerated.

Results

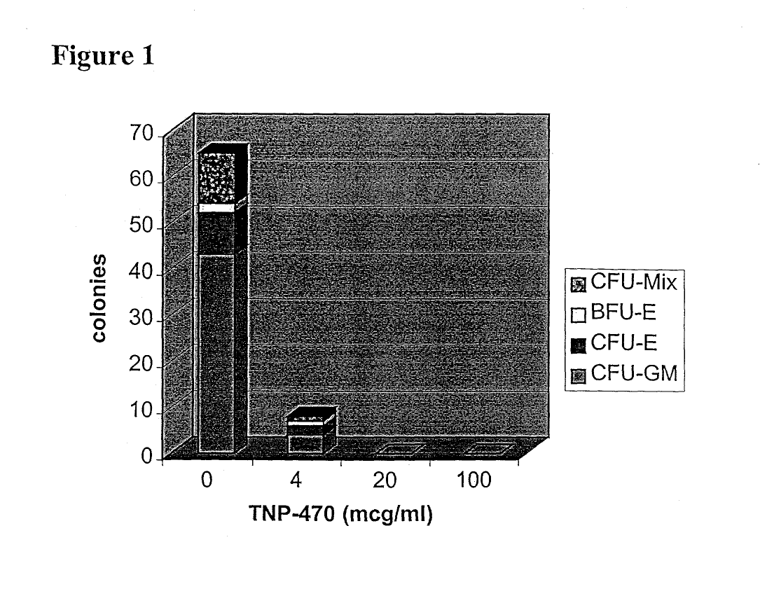

Effect of TNP-470 on bone marrow colony-forming cells. Although hematologic toxicity has not been described in the TNP-470 phase I trials (24, 25), there is one report of in vitro evidence of BM toxicity (16). In order to confirm this finding, we investigated the effect of relatively high concentrations of TNP-470 (0-100 μg/ml) on growth of human hematopoietic progenitors in standard methylcellulose culture. As shown in FIG. 1, inhibition of colony formation in the presence of TNP-470 was observed both for myeloid and erythroid colonies. TNP-470 (4 μg/ml) caused >80% inhibition of colony formation and higher doses caused complete inhibition of colony-forming cells. Although this assay is not necessarily predictive of in vivo BM toxicity, the result emphasizes the need to develop a pre-clinical model of stem cell transplantation to assess the effect of AIs on engraftment.

Validating the stem cell transplant model. The development of a model for stem cell transplant in which to test the effects of AIs on engraftment facilitated the identification of a dose of TBI that was lethal without stem cell rescue and a threshold stem cell dose that reliably provided engraftment. The lethal dose of radiation was determined by tail vein injection of 0 or 5×10⁶ BM cells after varying doses of TBI. As shown in Table 1, mortality with and without marrow support was investigated at TBI doses levels of 500 cGy (300 cGy followed by 200 cGy, or 300/200 cGy), 700 cGy (400/300 cGy) and 900 cGy (500/400 cGy). Mice given 900 cGy had a mortality rate of 80-100% in the absence of stem cell support and mortality rate of 0-12.5% when 5×10⁶ BM cells were infused.

TABLE 1
TBI dose with and without stem cell support¹

| TBJ dose in cGy | Mortality | Mortality |
|---|---|---|
| (fraction sizes) | (no support) | (marrow support) |
| 500 (300/200) | 10-25% | 0% |
| 700 (400/300) | 50-60% | 10% |
| 900 (500/400) | 100% | 0-12.5% |

¹Data indicate ranges of percent mortality in experimental groups given the indicated TBI dose with or without 5 × 10⁶ BM cells to support hematologic recovery. 8-10 recipient mice/group with 2 (500 cGy) or 3 (700 and 900 cGy) groups evaluated at each dose.

Having chosen the TBI dose of 900 cGy for further studies, a minimum stem cell dose that would reliably provide engraftment in most recipients was determined. Choosing such a threshold stem cell dose would increase the likelihood of demonstrating a small effect of TNP-470 on engraftment. For these experiments, engraftment was defined as >5% Tg+ B cells in the spleen or BM as detected by flow cytometry on day 28 post-stem cell infusion (day 28). Lethally irradiated mice were injected with BM doses ranging from 0 to 5×10⁶ cells in 1×10⁶ cell dose intervals. Mortality was determined by observation and engraftment of B cells was determined at each dose level by flow cytometry (Table 2). A cell dose of 2-3×10⁶ stem cells per mouse was found to have a mortality rate of 8-40% with a BM engraftment rate of 75-100%, thus assuring consistent engraftment at a minimum cell dose. The radiation and cell doses established a baseline that was used in TNP-470 experiments.

Relationship of BM cell dose to engraftment and mortality^{a}

| BM dose | | |
|---|---|---|
| (×10⁶ cells) | Mortality | Engraftment |
| 0 | 100% | NA^{b} |
| 0.5 | 60-80% | 0-20% |
| 1 | 40% | 100% |
| 2 | 8-37.5% | 75-100% |
| 3 | 0-17% | 100% |
| 5 | 0-20% | 80-100% |

^{a}Data indicate ranges of percent mortality in experimental groups given 900 cGy TBI followed by the indicated dose of BM cells. 5-8 recipient mice/group with 2-3 groups evaluated at each dose.
^{b}NA = not applicable.

Effect of TNP-470 on engraftment. Several dose levels of TNP-470 were explored to assess any effect on BM engraftment and engraftment kinetics. The dosing regimens are summarized in Table 3. Immediately following lethal irradiation and tail vein injection of Tg+ BM cells, recipient mice were given either TNP-470 or saline starting on day 0. Dose schedules were also varied from a single dose at the time of stem cell infusion to an initial dose on day 0 followed by administration of TNP-470 or saline 3 times a week (Table 3). Mice were initially sacrificed on days 28-32 at all dose levels. Comparative kinetics were then further analyzed by sacrificing groups of mice at day 21, day 24, and day 28 at dose level 3.

TABLE 3
TNP-470 Dose Levels

| | | Subsequent dose |
|---|---|---|
| Dose Level | Dose on Day 0 | and schedule |
| 1 | 20 mg/kg | 20 mg/kg t.i.w.^{a} |
| 2 | 100 mg/kg | None |
| 3 | 100 mg/kg | 20 mg/kg t.i.w. |
| 4 | 100 mg/kg | 100 mg/kg t.i.w. |
| Continuous | 10 mg/kg/week | |
| infusion | starting on day 1 | |

^{a}TNP-470 was given SQ t.i.w. (three times/week)

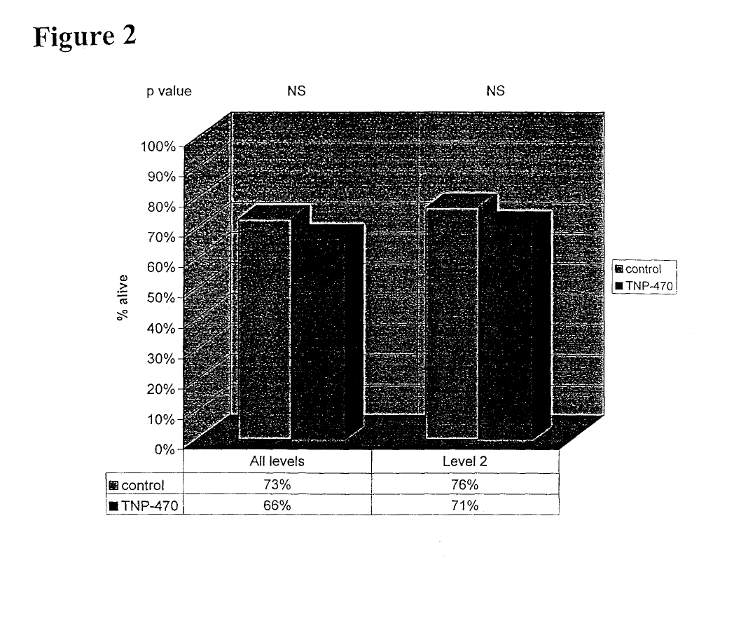

Overall survival at all dose levels was 73% for mice treated with placebo and 66% for TNP-470 treated animals (FIG. 2). At completion of the experiment, analysis of mortality at dose level 1 (20 mg/kg on day 0 and then thrice weekly) revealed that the survival rate was 57% for treated mice and 64% for control mice. Furthermore, for dose level 2, (100 mg/kg on day 0 only) 71% of treated mice survived to experiment completion while 76% of control mice were alive at day 28-32. This difference was not statistically significant. At the doses tested, these data provide no indication of a dose-dependent effect of TNP-470 on post-bone marrow transplant (BMT) survival. Toxicities overall were minimal, although the treated mice at dose level 4 (100 mg/kg 3×/week) experienced greater weight loss than the control animals and showed evidence of skin irritation at the injection sites.

Lymphocyte engraftment was not affected by treatment with TNP-470. When analyzed by flow cytometry to determine the percentage of B lymphocytes expressing the donor-origin transgenic IgM (B220+/IgM+), TNP-470 treated and control transplanted mice expressed similar percentages of Tg IgM+ cells in spleen and BM. (Table 4). Engraftment kinetics were then explored at dose level 3 (100 mg/kg on day 0, followed by 20 mg/kg 3 times/wk). Splenic reconstitution in treated animals at days 21, 24 and 28 was comparable to that of controls. BM engraftment for TNP-470 exposed mice was significantly better than controls at day 21, though this difference disappeared at day 24 and day 28 (Table 4).

TABLE 4
Lymphoid engraftment after stem cell rescue^{a}

| | Spleen | | Bone Marrow | |
|---|---|---|---|---|
| N | B220+/IgM+ | p | B220+/IgM+ | p |

Day 28 post-BMT

| | | | | | |
|---|---|---|---|---|---|
| Control | 54 | 11% ± 1.2^{b} | | 20% ± 1.4 | |
| All TNP-470 | 54 | 14% ± 1.4 | NS^{c} | 22% ± 1.8 | NS |
| dose levels | | | | | |
| Engraftment kinetics, | | | | | |
| dose level 3 | | | | | |
| Day 21 Control | 8 | 12% ± 4.2 | | 15% ± 2.5 | |
| Day 21 TNP-470 | 5 | 13% ± 2.8 | NS | 27% ± 4.1 | .03 |
| Day 24 Control | 6 | 22% ± 5.3 | NS | 28% ± 4.8 | NS |
| Day 24 TNP-470 | 6 | 31% ± 5.4 | NS | 25% ± 2.7 | NS |
| Day 28 Control | 10 | 9% ± 1.1 | NS | 23% ± 4.1 | NS |
| Day 28 TNP-470 | 14 | 11% ± 2.0 | NS | 28% ± 4.2 | NS |

^{a}Lymphoid engraftment at all TNP-470 dose levels as determined by flow cytometry is indicated in the upper half of the table. Engraftment kinetics at dose level 3 are indicated in the lower half of the table. Bone marrow and splenic reconstitution with donor-derived cells were measured at days 21, 24 and 28 post-BMT.
^{b}Mean ± SEM. Only the value indicated in bold is significantly different from control. Recipient mice were assayed for engraftment by detection of the IgM transgene derived from donor stem cells. The percent of B220+/Tg IgM+ cells in the lymphoid size/granularity gate are indicated.
^{c}NS, not significant

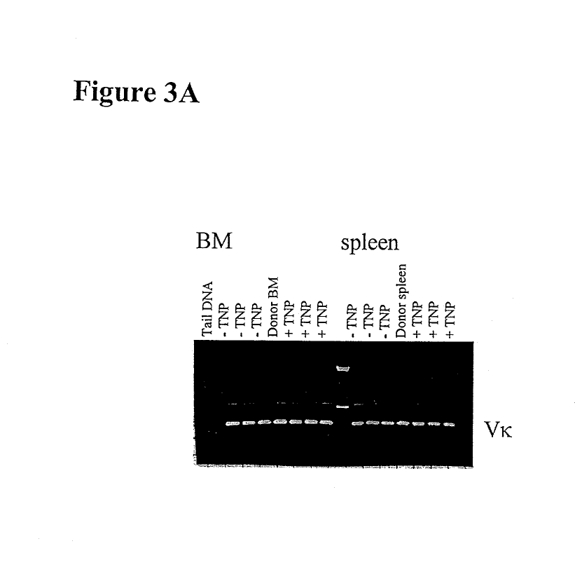

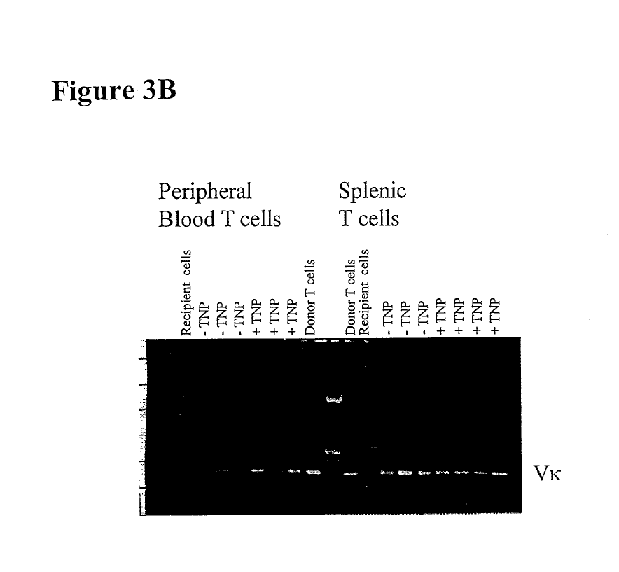

PCR analysis was performed on various cell populations to detect cells that carry the Tg. The transgene was detectable in any cell derived from the graft, regardless of lineage (B cells or non-B cells). In FIG. 3A, DNA from BM and splenocytes from transplanted mice treated with TNP-470 or saline was analyzed for the presence of the transgene by PCR. The transgene was detected in all samples analyzed, irrespective of treatment with TNP-470. Tail DNA from a Tg− animal provided a negative control, and BM and spleen from a Tg+ animal was used as a positive control. As seen in FIG. 3B, T cells were isolated from spleen and peripheral blood of transplanted animals one month after transplant and subjected to PCR detection of the Tg. For peripheral blood, T cells were isolated after Ficoll separation of the mononuclear cell fraction using the FACS Vantage cell sorter to sort CD3+ cells. For splenocytes, T cells were isolated using a mouse T cell isolation column. Cytometric analysis of the splenic T cells showed that the T cells were 92-95% CD3+ after column isolation (data not shown). Post-sort analysis of peripheral blood T cells was not possible because of the limited number of cells isolated. Again, repopulation of the T cell lineage with donor-derived cells was seen in both TNP-470-treated and control animals, with the Tg detected in all splenic T cell samples, 3/3 TNP-470-treated peripheral blood T cell samples, and 2/3 control peripheral blood T cell samples.

As summarized in Table 5, peripheral blood parameters were also assessed for hematologic recovery. No statistical differences were found in hemoglobin or platelet count between control and treated mice. Control mice, however, had a higher mean white blood cell count (6700/μl) than animals treated with intermittent TNP-470 (3600/μl); this difference in white blood cell count was statistically significant (p<0.04). Despite the statistical difference, the post-BMT white blood cell count reached by treated mice was adequate and within the normal range. Bone marrow cellularity was also assessed in the femurs of transplanted animals. At day 28 after dose level 3, there was no difference in marrow cellularity between TNP-470-treated transplanted mice, transplanted control mice, or untransplanted control mice.

TABLE 5
Recovery of peripheral WBC after stem cell rescue

| | N | WBC^{a} | P |
|---|---|---|---|
| Control | 12 | 6.3 ± 0.7^{b} | |
| TNP-470 | 10 | 3.6 ± 0.7 | .04 |
| intermittent dosing^{c} | | | |
| TNP-470 | 10 | 6.3 ± 0.4 | NS |
| continuous infusion^{d} | | | |
| PBS | 10 | 4.6 ± 0.3 | .03 |
| continuous infusion | | | |
| Untreated FVB | 10 | 7.1 ± 0.7 | NS |
| mice | | | |

^{a}White blood cell count in 10³/μL. WBC counts at day 28 post BMT in control animals and animals treated with TNP-470, continuous phosphate-buffered saline, as well as untransplanted untreated mice as a normal reference (FVB).
^{b}Mean ± SEM. Only the values indicated in bold are significantly different from control.
^{c}These animals received TNP-470 at dose level 3, SQ three times/wk, day 0 through day 28.
^{d}These animals received TNP-470 IP via Alzet pump at 10 mg/kg/wk.

Although TNP-470 was demonstrated to be effective in mouse xenograft models in treating established and early neuroblastoma tumors (12, 14) when given in an intermittent schedule, the current human clinical trials are including a continuous infusion component to overcome the short half-life of the drug (25). In order to reflect this dosing strategy, peritoneally implanted Alzet osmotic pumps were implanted in mice to deliver TNP-470 continuously over 14 days. In other studies, significantly lower total doses have been shown to provide similar anti-tumor effects and were tolerated in the continuous infusion setting (14). Based on these studies, a dose of 10 mg/kg/week given continuously was chosen as the highest dose that would not cause cachexia in transplanted and xenografted animals. In these experiments, pumps were implanted on day 1 prior to BMT. TBI and stem cell infusion occurred on day 0, and the animals were sacrificed on day 28. Lymphoid engraftment was assessed in these animals as above, and no differences were observed among TNP-470-treated animals, saline treated animals (both by continuous infusion), or transplanted animals with no pump implanted. As with the bolus dosing, no significant differences in hemoglobin or platelet recovery between TNP-470-treated and control animals were observed. In contrast to the bolus-dosed animals, however, no differences in WBC recovery were observed in the animals given TNP-470 by continuous infusion (Table 5) compared to animals that had been transplanted but had no pump implanted or normal (untreated and untransplanted) recipient mice. Among these animals, lower WBC counts were noted in the saline-treated animals (WBC=6300/μl in the TNP-470-treated mice vs. 4600/μl in the saline controls, p<0.03). Again, this difference, though statistically significant, was likely not clinically significant.

The present example shows that TNP-470 does not adversely impact engraftment after stem cell transplant and that full immune and hematologic reconstitution proceed uninterrupted. Accordingly, the method of the invention may provide a complimentary approach to the treatment of advanced pediatric solid tumors. Taken together with the xenograft model experience and the indication that angiogenesis inhibitors may work best when disease burden has been minimized, the data presented herein underscore the potential for therapeutic approaches in which anti-angiogenic agents are given in the post-transplant period to consolidate a remission and increase the likelihood of long-term disease control (58).

### EXAMPLE II

The following example sets forth a proposed therapeutic regimen that utilizes angiogenesis inhibitors as an adjunct to high-dose radiation with stem cell rescue in the treatment of cancer in humans. The administration of angiogenesis inhibitors in the context of minimal residual disease may decrease the likelihood of relapse and increase the three-year event-free survival probability for cancer patients.

Children with high-risk neuroblastoma treated with standard chemotherapy, radiation and surgery have at least an 80% likelihood of relapse. Even when treatment is augmented with autologous bone marrow transplantation and oral cis-retinoic acid, the likelihood of relapse is still 60% (2). However, the positive results obtained with cis-retinoic acid suggest that non-chemotherapeutic treatment at a time of minimal residual disease will improve outcome. Clearly, these data suggest that these patients would benefit substantially from the implementation of angiogenesis inhibitors as an adjunct to high-dose radiation with stem cell rescue. Other cancer patients, irrespective of age, who are in a state of minimal residual disease following other stem cell transplant modalities may also benefit from subsequent treatment with angiogenesis inhibitors.

Briefly, children with high-risk neuroblastoma may be treated with angiogenesis inhibitors following the induction and tandem transplant regimen reported in Grupp et al. (41). The administration of angiogenesis inhibitors in the setting of minimal residual disease provides an optimized therapeutic condition in which any remaining cancer cells are rendered most vulnerable to the biological effects of AIs. TNP-470, an example of an angiogenesis inhibitor that has shown promise in clinical studies, can be used as the angiogenesis inhibitor.

Children in a state of minimal residual disease can be administered TNP-470 at a dose of 60 mg/m², given during one-hour intravenous infusions, three times a week. This regimen of TNP-470 administration is considered the maximal tolerated dose (MTD) on the intermittent schedule most appropriate for pediatric patients on the intermittent schedule. Patients can initially be treated with TNP-470 starting at day +21 after stem cell transplant at a time when all patients should be fully engrafted and most patients should be ready for discharge. The initial treatment of patients with TNP-470 can, however, occur at earlier days post-transplant; a regimen for the initial administration of TNP-470 to patients at day +14, day +7, and day +1 post-transplant may be of utility in the treatment of such patients.

Although the utilization of TNP-470 is exemplified above, other angiogenesis inhibitors could be utilized. Moreover, if deemed efficacious, combinations of different angiogenesis inhibitors may be used for the treatment of patients with cancer.

This combined therapeutic approach may extend the duration of remission from disease and may potentially facilitate long-term complete remission. More specifically, the method of the present invention is expected to improve the prognosis of patients afflicted with the more severe types of cancer, including: relapses, advanced solid tumors, other high-risk solid tumors, and pediatric malignancies such as Ewing's sarcoma and neuroblastoma.

### REFERENCES

1. Matthay, K. K., et al., Med Pediatr Oncol. 31: 191, O-7, 1998.

2. Matthay, K. K., et al., N Engl J Med. 341: 1165-1173, 1999.

3. Rill, D. R., et al., Blood. 84: 380-383, 1994.

4. Brenner, M., et al., Lancet. 341: 85-86, 1993.

5. Freedman, A. S., et al., Blood. 94: 3325-3333, 1999.

6. O'Reilly, M. S., et al., Cell. 79: 315-328, 1994.

7. Folkman, J., Sci Am. 275: 150-154, 1996.

8. Folkman, J., Nat Biotechnol. 15: 510, 1997.

9. Parangi, S., et al., Proc Natl Acad Sci U S A. 93: 2002-2007, 1996.

10. Boehm, T., et al., Nature. 390: 404-407, 1997.

11. Kusaka, M., et al., Br J Cancer. 69: 212-216, 1994.

12. Katzenstein, H. M., et al., Clin Cancer Res. 5: 4273-4278, 1999.

13. Wassberg, E., et al., Pediatr Res. 41: 327-333, 1997.

14. Shusterman, S., et al., Med Ped Oncol. 35:511-673-676, 2000.

15. Cronin, F. E., et al., J Immunol. 161: 252-259, 1998.

16. Hasuike, T., et al., Eur J Haematol. 58: 293-294, 1997.

17. Matthay, K. K., et al., Eur J Cancer. 31A: 572-575, 1995.

18. Grupp, S. A., et al., J Clin Onc. 18: 2567-2575, 2000.

19. Grupp, S. A., et al., Med Pediatr Oncol. 35:696-700, 2000.

20. Ingber, D., et al., Nature. 348: 555-557, 1990.

21. Meitar, D., et al., J Clin Oncol. 14: 405-414, 1996.

22. Nagabuchi, E., et al., J Pediatr Surg. 32: 287-293, 1997.

23. Yanase, T., et al., Cancer Res. 53: 2566-2570, 1993.

24. Milkowski, D. M. and Weiss, R. A. TNP-470. In: Antiangiogenic Agents in Cancer Therapy, pp. 385-398. Teicher, B. A., ed. Humana Press, Totawa, N.J., 2000.

25. Bhargava, P., et al., Clinical Cancer Research. 5:1989-95, 1999.

26. Sobecks, R. M., et al., Bone Marrow Trans. 25:807-813, 2000.

27. Thomas, E. D., Seminars in Hematology. 36:95-103, 1999.

28. Duenas-Gonzalez, A., et al., Int J Gynecol Cancer 9:333-336, 1999.

29. Smith, M. L. and Newland, A. C., QJM 92:11-14,1999.

30. Patriarca, F., et al., Haematologica 85:269-274, 2000.

31. San Miguel, J. F., et al., Haematologica 84:36-58, 1999.

32. Anselmo, A. P., et al., Ann Hematol 79:79-82, 2000.

33. Hu, W. W., et al., Biol. Blood Marrow Transplant 6:58-69, 2000.

34. Tran, H. T., et al., Bone Marrow Transplant 26:463-470, 2000.

35. Abella, E. and Ravindranath, Y. Curr Oncol Rep 2:529-538, 2000.

36. Kollmannsberger, C., et al., Ann Oncol 11:553-559, 2000.

37. Steingrimsdottir, H. et al., Haematologica 85:832-838, 2000.

38. Sosman, J. A., et al., J Clin Oncol 19:634-644, 2001.

39. Margolis, J., et al., Seminars in Oncol 27:524-530, 2000.

40. Fisher, R. I., Seminars in Oncol 27:2-8, 2000.

41. Grupp, S. A. et al., J Clin Oncol 18:2567-2575, 2000.

42. Kletzel, M., et al., J Pediatr Hematol Oncol 20:49-54, 1998.

43. Grupp, S. A., et al., In: Transplantation in Hematology and Oncology II, Berdel, W. E., Buchner, T., Kienst, J., Jurgens, H., Ritter, J., Vormoor, J., eds., Springer-Verlag, Berlin: in press.

44. Lode, H. N., et al., Eur J Cancer 33:2024-2030, 1997.

45. Collins, R. H. Stem Cells 12:577-585, 1993.

46. Deplanque, G. and Harris, A. L., Eur J Cancer 36:1713-1724, 2000.

47. Folkman, J. Forum 9:59-62, 1999.

48. Shalinsky, D. R., et al., Clin Canc Res 5:1905-1917, 1999.

49. Los, M. and Voest, E. E., Seminars in Oncol 28:93-105, 2001.

50. Schirner, M., Cancer and Metastasis Rev 19:67-73, 2000.

51. Kruger, E. A. and Figg, W. D. Expert Opin on Invest Drugs 9:1383-1396, 2000.

52. Nagler, A. et al., Exp. Hematol 28:1096-1104, 2000.

53. Thomas, E. D., Stem Cells 12:539-544, 1994.

54. Saba, N., et al., Critical Rev in Oncology-Hematology 36:27-48, 2000.

55. Simnett, S. J., et al., Clinical and Laboratory Haematology 22:61-72, 2000.

56. Yoshida, A., et al., Histol & Pathol 14:1287-1294, 1999.

57. Shusterman, et al., Clin Cancer Res 7:977-984, 2001.

58. Stern J. W., et al., Clin Cancer Res 7:1026-1032, 2001.

What is claimed is:

1. An improved method for treatment of cancer in a patient who has undergone high-dose therapy with stem cell rescue transplantation, the improvement which comprises the administration to said patient of a therapeutically effective amount of an angiogenesis inhibitor at least during hematopoietic engraftment following said stem cell rescue transplantation.

2. The method of claim 1, wherein said cancer treatment includes high-dose chemotherapy.

3. The method of claim 1, wherein said cancer treatment includes high-dose radiation therapy.

4. The method of claim 1, wherein said cancer treatment includes a combination of high-dose chemotherapy and high-dose radiation therapy.

5. An improved method for treatment of cancer in a patient who has undergone high-dose therapy and stem cell rescue transplantation, the improvement which comprises administration to said patient of a therapeutically effective amount of an angiogenesis inhibitor at least during hematopoietic engraftment following said stem cell rescue transplantation, the angiogenesis inhibitor comprising a compound, including pharmaceutically acceptable salts, having the formula: 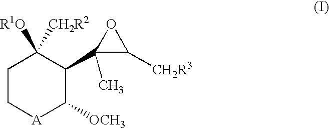
wherein:
R¹ represents hydrogen;
R² represents halogen, N(O)ₘR^{a}R^{b}, S(═O)ₙR^{a}, N⁺R^{a}R^{b}R^{c}X⁻, or S⁺R^{a}R^{b}X⁻;
R^{a}, R^{b}, and R^{c} each being independently selected from the group consisting of hydrogen, an unsubstituted or substituted hydrocarbon, and an unsubstituted or substituted heterocyclic group;
alternatively, R¹ and R² together represent a chemical bond;
R³ represents an unsubstituted or substituted 2-methyl-1-propenyl or isobutyl group;
A represents >CH—OR⁴, >CH—NR⁵R⁶, or >C═O
R⁴ representing hydrogen; unsubstituted or substituted acyl; unsubstituted or substituted alkyl; unsubstituted or substituted aryl; unsubstituted or substituted carbamoyl; unsubstituted or substituted benzenesulfonyl; unsubstituted or substituted alkylsulfonyl; unsubstituted or substituted sulfamoyl; unsubstituted or substituted alkoxycarbonyl; or unsubstituted or substituted phenoxycarbonyl;
R⁵ and R⁶ each independently representing hydrogen, unsubstituted or substituted acyl; unsubstituted or substituted alkyl; unsubstituted or substituted aryl; unsubstituted or substituted carbamoyl; unsubstituted or substituted alkoxycarbonyl; or unsubstituted or substituted phenoxycarbonyl;
X⁻ is a counteranion;
m is an integer 0 or 1; and
n is an integer 0, 1, or 2.

6. The method according to claim 5, wherein said angiogenesis inhibitor comprises a compound of the formula:

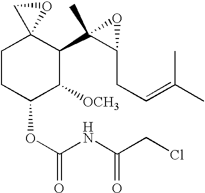

7. A method according to claim 1, wherein said angiogenesis inhibitor is administered intravenously.

8. A method according to claim 1, wherein said angiogenesis inhibitor is administered by continuous infusion.

9. A method according to claim 1, wherein TNP-470 is administered as the angiogenesis inhibitor in the amount of 70 mg/m², three times weekly.

10. A method according to claim 1, wherein said angiogenesis inhibitor is further administered prior to hematopoietic engraftment.

11. A method according to claim 1, wherein said angiogenesis inhibitor is further administered after hematopoietic engraftment.
